(19) United States
(12) Patent Application Publication
(10) Pub. No.: US 2006/0264813 A1
(43) Pub. Date: Nov. 23, 2006

(54) Aerated and deaerated intestinal irrigation apparatus

(76) Inventor: Wolfgang Herrmann, Eichelsbach (DE)

Correspondence Address: KREMBLAS, FOSTER, PHILLIPS & POLLICK, 7632 SLATE RIDGE BOULEVARD, REYNOLDSBURG, OH, 43068, US

(21) Appl. No.: 11/406,891

(22) Filed: Apr. 19, 2006

(30) Foreign Application Priority Data
Apr. 27, 2005 (EP) EP05103460.1

(51) Int. Cl. A61M 3/00

(52) U.S. Cl. 604/43

(57) ABSTRACT

An aeration and deaeration system for an intestinal irrigation apparatus, with an intestinal drainage line, the inlet of which can be connected to the outlet of a human or animal intestine for draining the flushed-out intestinal content, with the intestinal drainage line being closable by means of a shut-off device disposed on the outlet side for retaining fluid in the intestine and with the segment of said intestinal drainage line between its inlet and outlet being coupled to at least one aeration and deaeration system which is designed so as to be able to automatically close beginning at a specific pressure threshold value in the intestinal drainage line.

### BACKGROUND OF THE INVENTION

1. Field of the Invention

The subject matter of the present invention relates to an aeration and deaeration system for an intestinal irrigation system, with an intestinal drainage line, the inlet of which can be connected to the outlet of a human or animal intestine for draining the flushed-out intestinal content.

2. Description of the Related Art

The subject matter of the present invention also relates to an intestinal irrigation apparatus with an intestinal feed line which can be connected to a human or animal intestine for feeding in irrigation fluid and with an intestinal drainage line which can be connected to the intestine for draining the flushed-out intestinal content.

Aerated and deaerated intestinal irrigation systems, in particular for medical and therapeutic, use have been known for a long time (see DE-U 296 10 226 or DE-U 20 2004 015 358). Such an apparatus usually comprises a feed line via which water is introduced under low pressure into the intestine by means of a catheter or a speculum. The process of introducing the irrigation fluid into the intestine is normally aided by the therapist by means of massages. The massages also serve to loosen incrustations and deposits to ensure that these are removed from the intestine as the irrigation fluid is drained off. The catheter or the speculum is fitted with a connecting piece for a drainage line which is led back to the apparatus housing where it discharges into an inspection tube for monitoring the draining irrigation fluid. A shut-off device for retaining the water is generally disposed within the drainage line upstream or downstream of the inspection tube. Downstream of the inspection tube, the drainage line continues and is connected to an outlet drain that is built in on site. Via this outlet drain, the discharged irrigation fluid is discharged into the wastewater system.

In addition, such an apparatus generally also comprises an inlet for cold water and for hot water, from which a mixture of water can be drawn, the temperature of which water can be adjusted by means of a temperature-control device so as to be pleasant to the patient. In addition, the apparatus also comprises a pressure regulator, by means of which the pressure can also be adjusted to the requirements of the patient. Furthermore, to monitor the temperature and the pressure of the water, generic apparatuses also comprise a temperature indicator and a pressure indicator.

During the treatment of a patient with such an intestinal irrigation apparatus, air in the intestine of patients, in particular those who are being treated for flatulence, can cause an unpleasant feeling of pressure. This air furthermore prevents irrigation fluid from being flushed in and thus prolongs the treatment process. This problem is further compounded by air which is introduced into the intestine of the patient along with the irrigation fluid. During the treatment, the therapists often remedy this situation by intermittently and briefly pulling out the drainage tube, thereby equilibrating the pressure and thus ensuring the evacuation of air. The disadvantage, however, is that for the therapist to be able to do this, the patient must move into a lateral position, which, in view of the speculum that has been inserted into the anus of the patient, is inconvenient, especially for old and/or ill individuals, as well as embarrassing. In addition, during such a manual pressure equilibration, it is not possible to avoid the discharge of malodorous gases and small quantities of irrigation fluid. On the one hand, this leads to an odor nuisance during the treatment and, on the other hand, the discharge of such contaminated irrigation fluid entails hygienic risks. Furthermore, because of the unavoidable soiling of the treatment table, the cleaning expenses increase, which increases the personnel cost for the treatment. On top of all this, due to the delays during the manual deaeration, the length of treatment time increases considerably, which has a negative effect on the efficiency of the treatment method, which in turn increases the cost. Once sufficient fluid has been introduced into the intestine, the shut-off device of the drainage line is opened during the treatment, and due to the suction effect of the lower-lying drain, the intestine is emptied, with the intestinal deposits and incrustations being swept along in the irrigation fluid. This, however, may give rise to the problem that solid components contained in the irrigation fluid may at least temporarily clog the drainage line, with the effect that the water containing fecal components can, if at all, drain off only with difficulty. Thus, an improvement of the drainage line of an intestinal irrigation apparatus is desirable.

The problem frequently encountered during an intestinal irrigation—i.e., that the fluid which has been suctioned off by the intestinal irrigation apparatus and which contains intestinal components comes to a stop, for example, in an intestinal content inspection tube, which, as a result, interrupts the suction drainage procedure—has been addressed in the utility model patent publication DE-U 20 2004 016 919. To solve the problem, this publication proposes a device for aerating the intestinal irrigation apparatus by means of an air inlet valve which is coupled to the drainage line that leads to the inspection tube. When aeration is not needed, the valve is in a starting position in which the valve body is pressed against a valve seat by means of spring pressure so as to seal the valve. If the irrigation procedure comes to a standstill, which the operator can observe from the outside in the inspection tube, said operator need do nothing more than briefly press the operator button of the air inlet valve, which causes said valve to be opened against the spring pressure. Because of the suction effect, air is introduced into the obstruction in the drainage line, without allowing malodorous gases to exit. In the open position of the valve in which the operator button of said valve is pressed, however, it is not possible to eliminate the risk that contaminated water or fluid containing intestinal contents may exit via the valve throat area. This risk arises, e.g., if the operator button is pressed by mistake or by accident.

Thus, the problem to be solved by the present invention is to increase the functional safety and the operating safety of an intestinal irrigation apparatus. To solve this problem, the present invention proposes the aeration and deaeration system disclosed in claim 1 and the intestinal irrigation apparatus with a suitable aeration and deaeration system disclosed in claim 10 or 11. Useful optional embodiments follow from the dependent claims.

### BRIEF SUMMARY OF THE INVENTION

By means of the shut-off device which, according to the present invention, is disposed on the outlet side in the intestinal drainage line, the irrigation fluid can be made to back up into the colon. During this back-up, the air inside the patient or the air from the tubular system of the intestinal irrigation apparatus is frequently backed up as well. This can lead to excessive flatulence of the patient. This problem is solved by the deaeration component of the aeration and deaeration system. If, during the back-up and deaeration cycle, fluid containing flushed-out intestinal content floods into the line leading to the air relief valve, the risk of contamination of the surrounding area is eliminated by the automatic shut-off mechanism which is integrated into the aeration and deaeration system and which is activated beginning at a specified pressure threshold value that corresponds to the inadmissible rise of the fluid level prior thereto after the air of the irrigation system has exited. At any other time, the aeration and deaeration system is in a permanent, invariably bidirectionally opened position which allows an automatic deaeration, i.e., without the manual intervention of the therapist.

To ensure a reliable discharge of air or gases from the drainage system of the intestinal irrigation apparatus, a useful embodiment of the present invention proposes that the connecting channel leading to the aeration and deaeration system be connected to the highest point of the drainage line. This utilizes the well-known effect that air or gas bubbles in fluids have the propensity to rise upward.

The operating reliability can be increased and the risk of odor nuisances can be decreased if, according to another embodiment of the invention, the aeration and deaeration system is disposed inside an apparatus housing and, in particular, is completely enclosed therein.

To ensure that the discharge of irrigation fluid is reliably prevented, it is important that the device for the air evacuation of the drainage line operates automatically, which means that said device must preferably be provided with means for a permanent communication with the ambient air so that a pressure equilibration can be produced; at the same time, however, it must also be able to immediately close the opening as soon as the risk of exiting water arises. This is implemented according to the present invention in that a valve is used, which valve is open to air but immediately stops rising water.

To improve the reduced discharge efficiency in cases in which fecal matter is contained in the exiting irrigation fluid, an increase in the discharge quantity and an improvement of the discharge characteristics can be ensured according to the present invention by feeding air into the drainage line while the irrigation fluid is being drained off. As known from the prior art, the introduction of air or the aeration of tubular lines increases [sic; leads to] an increased draining capacity by decreasing the formation of vorticity. According to an alternative embodiment of the present invention, a device for aerating the drainage line is therefore disposed on the drainage line upstream of the shut-off and control device. When the shut-off or control device is opened, a suction effect is produced in the device for aerating the drainage line, which suction effect leads to the entrainment of small air bubbles and thus contributes to the improvement of the draining efficiency. To prevent irrigation fluid from exiting, the aeration device is again designed so that in cases in which water rises into the aeration device, said aeration device is shut off, while it is otherwise open so that air can be fed into it. This shut-off action also takes place automatically and does not require any interaction [sic; intervention] the therapist.

An especially useful further development of the present invention proposes an intestinal irrigation apparatus into which devices for the aeration and deaeration are integrated in a combined form. In this manner, the advantages of an aeration and deaeration of the drainage device can be combined while creating synergistic effects. To implement this combination proposed by the invention, a useful embodiment proposes that an air inlet and air relief valve be used, which valve comprises a floating body that can be moved by means of fluid pressure against a valve seat of a flow-through valve housing. In an improved variation of this embodiment, the air inlet and air relief valve is disposed in or on the irrigation device in such a way that the floating body, as a result of its intrinsic weight, can be automatically moved away from the valve seat. As an alternative, it is also conceivable to use spring elements to maintain the open valve position. This type of automatic air inlet and air relief valve is known from the Utility Model DE-U 296 06 767. Also suitable as air inlet and air relief valves are tube aerators known from the prior art, for example, of the firm of Seppelfricke Armaturen GmbH & Co., D-45881 Gelsenkirchen, Model No. 8171 and 8172, Type D.

To assure that no irrigation fluid can spill around the device for aerating and evacuating air from the drainage line, another useful further improvement of the invention additionally provides for a collecting or overflow vessel. Thus, in cases in which small quantities of irrigation fluid are exiting, it is possible to reliably prevent these from entering the housing of the intestinal irrigation apparatus unnoticed. This once more further increases the operating safety of the apparatus considerably and simplifies the use of the apparatus for the therapist. From this overflow vessel, a line can be advantageously run to the drainage line, thereby ensuring that potentially exiting small quantities of irrigation fluid are reliably carried off in the direction of the wastewater system. This line to the drainage line can be advantageously disposed downstream of the shut-off and control device, thereby ensuring that even in cases of a closed shut-off and control device the potentially exiting irrigation fluid is reliably carried off.

In an especially advantageous further development, the devices for aerating and evacuating air in the intestinal irrigation apparatus according to the present invention can be designed in integrated form. In such an embodiment, the advantages and properties of an automatic aeration and deaeration surprisingly complement one another. Thus, the combined aeration and deaeration system may still allow unpleasant odors to exit during the air evacuation of the intestine or the drainage line and to escape into the inside of the intestinal irrigation apparatus; however, since, because of the entrained and thus removed air, the aeration function develops a certain suction effect, and unpleasant odors are again drawn in and thus removed before they can exit from the housing of the intestinal irrigation apparatus. In this manner, the developing unpleasant odors can be automatically removed even before they can develop into a nuisance for the patient and the therapist.

### BRIEF DESCRIPTION OF THE SEVERAL VIEWS OF THE DRAWINGS

The drawings below illustrate a preferred example of an embodiment of the present invention, without however intending in any way to restrict the invention to said drawings, and with many alternative embodiments and (sub)combinations that fall within the scope and nature of the invention being conceivable. As can be seen:

FIG. 1 shows a schematic representation of an irrigation apparatus according to the present invention with a patient lying on the patient treatment table;

FIG. 2 shows a schematic functional representation of the inside of the intestinal irrigation apparatus seen in FIG. 1, and

FIG. 3 shows a partial axial section of an air inlet and air relief valve that is suitable for use in an intestinal irrigation apparatus according to the present invention.

### DETAILED DESCRIPTION OF THE INVENTION

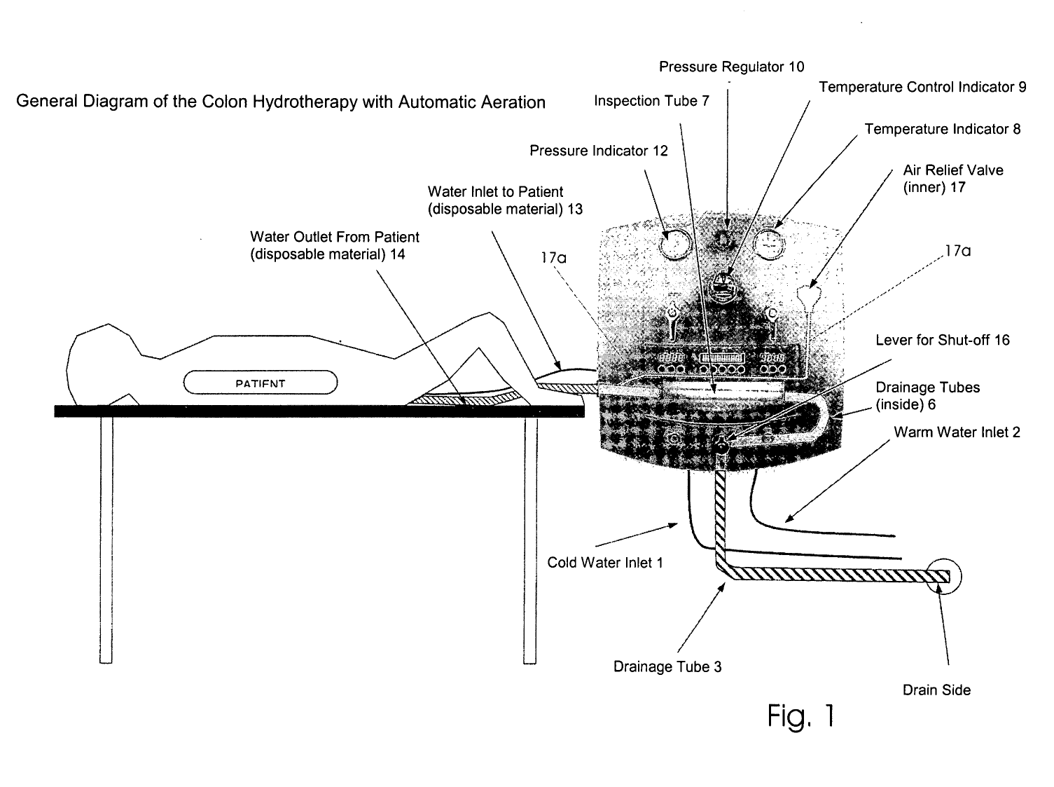

FIG. 1 shows a schematic representation of the configuration of a treatment room with an intestinal irrigation apparatus according to the present invention. The intestinal irrigation apparatus is mounted at approximately the same level as the treatment table on which the patient lies and is connected via inlets 1 and 2 to the cold and warm water line, respectively. A drainage tube 3 leads from the wastewater outlet of the intestinal irrigation apparatus to an outlet drain that is built in on site. By means of a temperature-control device 9, the water fed in via inlets 1 and 2 is brought to a water temperature that is pleasant to the patient, which water temperature can be monitored by means of the temperature indicator 8. By means of the pressure regulator 10, the pressure of the incoming water can be set so as to be comfortable for the patient and can also be monitored via the pressure indicator 12. Through the water inlet 13 which is made of a disposable material, water is delivered to the patient. Once the intestine of the patient has been sufficiently filled and cleaned by means of a supporting massage, the inlet is closed and the outlet is opened by means of the shut-off lever 16. The water present in the intestine flows through the water drain 14 which is also made of a disposable material to the inside drainage tube 6 and from there via the inspection tube 7 to the drainage tube 3 and via the outlet drain 18a which is built in on site into the wastewater system. Line 17a (shown as a broken line in the figure) in inside drainage tube 6 which leads to the deaeration device 17 is located between the connecting piece of the water drain 14 on the apparatus and the inspection tube 7. Potentially developing air in the intestine and/or in the drainage tube can escape via this air relief valve 17.

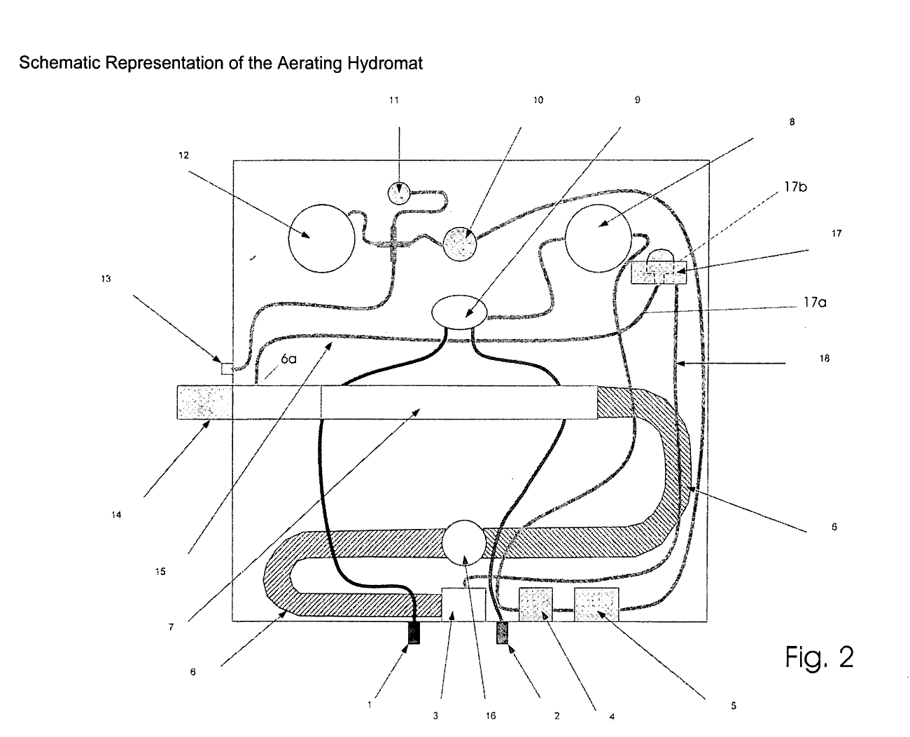

FIG. 2 shows a schematic representation of the internal setup of an intestinal irrigation apparatus according to the present invention. After the water has entered the apparatus via inlets 1 and 2, it is conducted from there via certain lines to the temperature-control device 9. From the temperature-control device 9, the water is again conducted via a line to the temperature indicator 8, by means of which the therapist can adjust the temperature so as to be pleasant for the patient. From the temperature indicator 8, the water is conducted via another line to a pressure reducing valve 4 which reduces the water pressure prevailing in the building network to the maximum treatment pressure. Directly downstream of the pressure reducing valve, an excess pressure shut-off valve 5 is disposed inside the line, which excess pressure shut-off valve, as an additional safety measure, interrupts the line to the patient if excess pressure builds up. From this excess pressure shut-off valve 5, another line leads to the pressure regulator 10, by means of which the pressure of the incoming water can be adjusted so as to be pleasant for the patient. Disposed downstream of the pressure regulator 10 by means of branching-off lines are a pressure indicator 12 and a pressure monitor 11 as an additional safety measure against excess pressure. From the group of pressure-control devices, the water is conducted via yet another line to connecting piece 13 to which the inlet line to the patient can be connected. On the connecting piece 14, the drainage line coming from the patient is connected. In the following section of the inside drainage line 6, the inlet to the air inlet and air relief valve 17 is connected in the direction of flow downstream of the inspection tube 7. The air inlet and air relief valve 17 is fitted with an overflow or collecting vessel 17b which can hold potentially exiting irrigation fluid that is contaminated with fecal matter. From this collecting vessel, yet another line 18 leads to the outlet connecting piece 3 which is connected via the drainage tube with the outlet drain 18a which is built in on site. In the area in which valve 17 with the inside drainage tube 6 is connected, the inspection tube 7 is disposed, by means of which inspection tube the therapist can inspect and monitor the flushed-out components of the irrigation fluid. Downstream of the inspection tube 7, the inside drainage tube 6 leads via the shut-off and control device 16 to the discharge connecting piece 3 or to the outlet drain 18a which is built in on site.

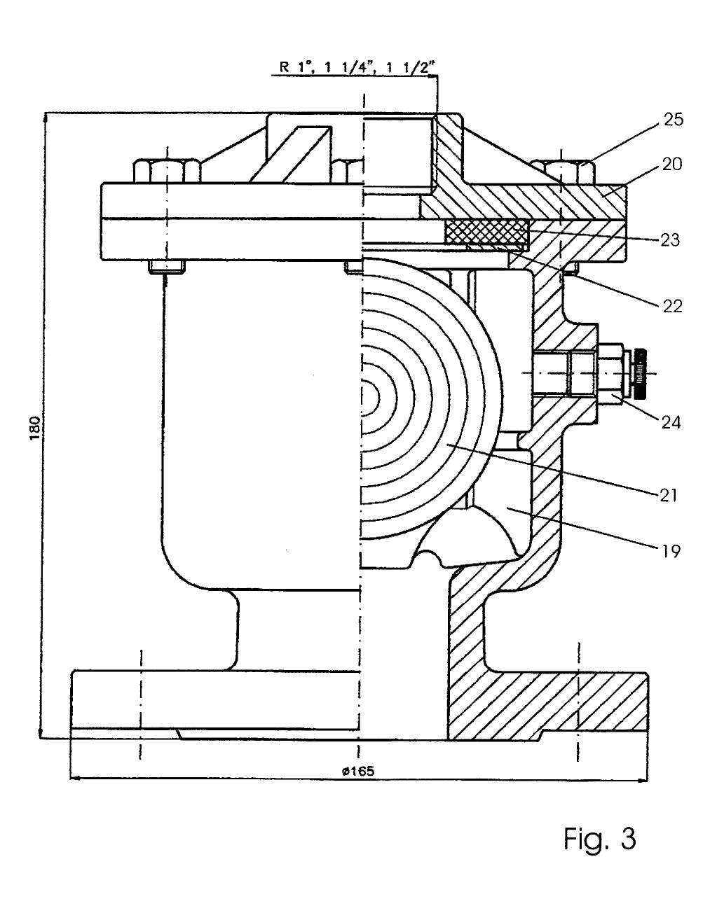

FIG. 3 shows an air inlet and air relief valve which can be used in an intestinal irrigation apparatus according to the present invention. This valve comprises the valve housing 19 which is designed so it can be connected to a tubular line. Following the connecting area which can be seen at the bottom of the drawing, there is a widened circular flow area in which a ball float 21 is located. The housing 19 of the air inlet and air relief valve is open at the top to allow the ball float 21 to be easily introduced into the circular flow area. After the ball float has been introduced, a cover 20 is screwed onto the upper opening of the valve housing 19. Cover 20 is screwed on by means of screws 25. Located in the center of cover portion 20 is an opening through which air can escape from the inside of the valve housing 19. In the valve body 19 around the opening in the valve cover 20 on the side of the circular flow area, a plain washer 22 and a rubber packing ring 23 are screwed in. This air inlet and air relief valve is mounted in the housing of the irrigation apparatus in such a way that the cover portion 20 is at the top. As a result, due to the force of gravity, the ball float 21 is located at the lower end of the circular flow area of the valve housing 19. The weight of the ball float 21 is selected to ensure that the exiting air is generally not able to push said ball float against the force of gravity onto the rubber packing ring 23. Thus, when water rises in the tube which is located at a level lower than the air inlet and air relief valve, the ball float 21 floats upward and presses itself against the rubber packing ring 23. As a result, the air inlet and air relief valve is rendered impermeable to the water and shuts off against the exiting water. When the ball float 21 is not pressed against the rubber packing ring 23, air can bidirectionally, i.e., both for aeration and for deaeration, flow through the valve.

It is obvious that other differently constructed air inlet and air relief valves can be used in an intestinal irrigation apparatus according to the present invention. In all cases, however, it is useful to ensure an automatic activation at least when the water is rising.

### List of Reference Numbers

1 Cold water inlet

2 Warm water inlet

3 Drainage tube

4 Pressure reducing valve for the maximum treatment pressure

5 Shut-off valve at excess pressure

6 Drainage tubes (inside)

6a Connecting point

7 Inspection tube

8 Temperature indicator

9 Temperature control device

10 Pressure regulator for the incoming treatment water

11 Pressure monitor for excess pressure (250 mbar)

12 Pressure indicator for the treatment pressure

13 Connecting piece for the treatment water (in the direction of the patient)

14 Connecting piece for the wastewater tube (coming from the patient)

15 Tube from the inspection tube to the air relief valve

16 Shut-off lever to retain patient water

17a Line to the air relief valve

17 Air relief valve with collecting vessel

17b Collecting vessel

18 Tube from the collecting vessel to the connecting piece for the outlet drain

18a Outlet drain built in on site

19 Valve housing

20 Cover

21 Ball float

22 Plain washer

23 Rubber packing ring

1. An aeration and deaeration system for an intestinal irrigation apparatus, with an intestinal drainage line (6), the inlet of which can be connected to the outlet of a human or animal intestine for draining the flushed-out intestinal content, characterized in that the intestinal drainage line (6) can be closed by means of a shut-off device (16) disposed on the outlet side for retaining fluid in the intestine and that, in its segment between the inlet and the outlet, said intestinal drainage line is connected to at least one aeration and deaeration system (17, 17a) which is designed so as to be able to automatically close beginning at a specific pressure threshold value in the intestinal drainage line (6).

2. The system as in claim 1, wherein the intestinal drainage line (6) runs so as to deviate from a horizontal line, characterized in that, with respect to the horizontal line, the connecting point (6a) of the intestinal drainage line (6) for the aeration and deaeration system (17, 17a) is disposed at the highest point of the intestinal drainage line (6).

3. The system as in claim 1, characterized in that the aeration and deaeration system (17, 17a) is disposed within an apparatus housing.

4. The system as in claim 1 characterized in that the aeration and deaeration system (17, 17a) is designed as a valve (19-25) that is bidirectionally open for the aeration and deaeration, which valve is in communication with the ambient air, on the one hand, and responding to the pressure in the intestinal drainage line (6), on the other hand, so as to automatically close beginning at a specified pressure threshold value.

5. The system as in claim 4, characterized in that the valve (19-25) is implemented in the form of a floating body (21) which, by means of fluid pressure, can be moved against a valve seat (20, 22, 23) of a flow-through valve housing (19) and which, by means of spring pressure and/or its intrinsic weight, can be moved away from the valve seat (20, 22, 23).

6. The system as in claim 1, characterized in that the aeration and deaeration system (17, 17a) can be implemented by means of a tube aerator.

7. The system as in claim 1, characterized in that the aeration and deaeration system (17, 17a) comprises a collecting vessel (17b) for exiting fluid.

8. The system as in claim 7, characterized in that the collecting vessel (17b) comprises a discharge line (18) which runs wholly or partially vertical to the intestinal drainage line (6), into which it discharges.

9. The system as in claim 8, characterized in that in the direction of flow downstream of the shut-off device (16), the discharge line (18) discharges into the outlet region of the intestinal drainage line (6).

10. An intestinal irrigation system with an intestinal feed line (13) which can be connected to a human or animal intestine for feeding in irrigation fluid and with an intestinal drainage line (6) which can be connected to the intestine for draining the flushed-out intestinal content, characterized in that the intestinal drainage line (6) can be closed by means of a shut-off device (16) which is disposed on the outlet side for retaining fluid in the intestine and that, in its segment between the inlet and the outlet, said intestinal drainage line is connected to at least one aeration and deaeration system (17, 17a) which is designed so as to be able to automatically close beginning at a specific pressure threshold value in the intestinal drainage line (6).

11. An intestinal irrigation apparatus in accordance with claim 1, and further comprising an intestinal feed line (13) which can be connected to a human or animal intestine for feeding in irrigation fluid, the intestinal drainage line (6) connectable to the intestine for draining the flushed-out intestinal content, and a shut-off device (16) which is disposed in an outlet segment of the intestinal drainage line (6) for retaining fluid in the intestine.
